(19) United States
(12) Patent Application Publication
(10) Pub. No.: US 2006/0183784 A1
(43) Pub. Date: Aug. 17, 2006

(54) Human growth hormone antagonists

(75) Inventor: Andrea G. Cochran, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/401,821

(22) Filed: Apr. 10, 2006

Related U.S. Application Data
(63) Continuation of application No. 10/172,247, filed on Jun. 14, 2002, now abandoned.
(60) Provisional application No. 60/298,358, filed on Jun. 15, 2001.

(51) Int. Cl. A61K 31/4184; A61K 31/404

(52) U.S. Cl. 514/394; 514/414; 514/415

(57) ABSTRACT

A method is disclosed for treating disorders in which human growth hormone is implicated by administering to a mammal an effective amount of an antagonist according to the general formula (I) 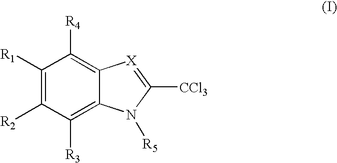
wherein X, R₁, R₂, R₃, R₄ and R₅ are as defined herein.

This is a non-provisional application claiming priority to non-provisional application Ser. No. 10/172,247, filed 14 Jun. 2002, and to provisional application No. 60/298,358, filed 15 Jun. 2001, the entire disclosures of which are hereby incorporated by reference.

### FIELD OF THE INVENTION

This invention relates to small molecule antagonists of human growth hormone (hGH) useful to treat hGH disorders, including methods of treatment and kits.

### BACKGROUND OF THE INVENTION

hGH participates in much of the regulation of normal human growth and development. This 22,000-dalton pituitary hormone exhibits a multitude of biological effects, including linear growth (somatogenesis), lactation, activation of macrophages, and insulin-like and diabetogenic effects, among others (Chawla, Annu. Rev. Med., 34:519 (1983); Edwards et al., Science, 239:769 (1988); Isaksson et al., Annu. Rev. Physiol., 47:483 (1985); Thorner and Vance, J. Clin. Invest., 82:745 (1988); Hughes and Friesen, Annu. Rev. Physiol., 47:469 (1985)). These biological effects derive from the interaction between hGH and specific cellular receptors.

Growth hormone deficiency in children leads to dwarfism, which has been successfully treated for more than a decade by exogenous administration of hGH. There is also interest in the antigenicity of hGH to distinguish among genetic and post-translationally modified forms of hGH (Lewis, Ann. Rev. Physiol., 46:33 (1984)), to characterize any immunological response to hGH when it is administered clinically, and to quantify circulating levels of the hormone.

hGH is a member of a family of homologous hormones that include placental lactogens, prolactins, and other genetic and species variants of growth hormone (Nichol et al., Endocrine Reviews, 7:169 (1986)). hGH is unusual among these in that it exhibits broad species specificity and binds to either the cloned somatogenic (Leung et al., Nature, 330:537 (1987)) or prolactin (Boutin et al., Cell, 53:69 (1988)) receptor.

The cloned gene for hGH has been expressed in a secreted form in E. coli (Chang et al., Gene, 55:189 (1987)) and its DNA and amino acid sequences have been reported (Goeddel et al., Nature, 281:544 (1979); Gray et al., Gene, 39:247 (1985)). The three-dimensional folding pattern for porcine growth hormone (pGH) has been reported at moderate resolution and refinement (Abdel-Meguid et al., Proc. Natl. Acad. Sci. USA, 84:6434 (1987)). The receptor and antibody epitopes of hGH have been identified by homolog-scanning mutagenesis and alanine-scanning mutagenesis in Cunningham et al., Science, 243: 1330-1336 (1989) and Cunningham and Wells, Science, 244: 1081-1085 (1989).

There are a large number of high-resolution structures that show the molecular details of protein-protein interfaces (for reviews, see Argos, Protein Eng., 2:101-113 (1988); Janin et al., J. Mol. Biol., 204:155-164 (1988); Miller, Protein Eng., 3: 77-83 (1989); Davies et al., Annu. Rev. Biochem., 59:439-473 (1990)). These define contact residues, but not the energetics for them nor do they show how docking occurs. A comprehensive understanding of the role of contact residues in affecting association and dissociation is fundamental to molecular recognition processes, and is practically important for rational protein and drug design.

Perhaps the best characterized hormone-receptor complex is that between hGH and the extracellular domain of its receptor (hGHbp). For a review, see Wells and De Vos, Annu. Rev. Biophys. Biomol. Struct., 22:329-351 (1993). High-resolution structural and mutational analysis (Cunningham and Wells, supra; Cunningham et al., Science, 254:821-825 (1991)) and structural analysis (De Vos et al., Science, 255: 306-312 (1992); U.S. Pat. No. 5,506,107) has shown that one molecule of hGH binds two receptor molecules sequentially using distinct sites on the hormone, called Sites 1 and 2.

A number of naturally occurring mutants of hGH have been identified. These include hGH-V (Seeberg, DNA, 1: 239 (1982); U.S. Pat. Nos. 4,446,235; 4,670,393; and 4,665,180) and 20K hGH containing a deletion of residues 32-46 of hGH (Kostyo et al., Biochem. Biophys. Acta, 925:314 (1987); Lewis et al., J. Biol. Chem., 253:2679 (1978)).

One investigator has reported the substitution of cysteine at position 165 in hGH with alanine to disrupt the disulfide bond that normally exists between Cys-53 and Cys-165 (Tokunaga et al., Eur. J. Biochem., 153:445 (1985)). This single substitution produced a mutant that apparently retained the tertiary structure of hGH and was recognized by receptors for hGH.

Another investigator has reported the in vitro synthesis of hGH on a solid resin support. The first report by this investigator disclosed an incorrect 188 amino acid sequence for hGH (Li et al., J. Am. Chem. Soc., 88:2050 (1966); U.S. Pat. No. 3,853,832). A second report disclosed a 190-amino acid sequence (U.S. Pat. No. 3,853,833). This latter sequence is also incorrect. In particular, hGH has an additional glutamine after position 68, a glutamic acid rather than glutamine at position 73, an aspartic acid rather than asparagine at position 106, and an asparagine rather than aspartic acid at position 108.

The structure of amino-terminal methionyl bovine growth hormone (bGH) containing a spliced-in sequence of hGH including histidine 18 and histidine 21 has been shown (U.S. Pat. No. 4,880,910). Additional hGH variants and anti-GH receptor antibodies are described in, e.g., U.S. Pat. Nos. 5,506,107 and 6,040,136; and WO 94/10994.

It has previously been shown that monovalent phage display (Bass et al., Proteins, 8: 309-314 (1990)) can be used to improve the affinity of Site 1 in hGH for the hGHbp (Lowman et al., Biochemistry, 30:10832-10838 (1991). Modest improvements in binding affinity (3 to 8-fold tighter than wild-type hGH) were produced by sorting three independent libraries each mutated at four different codons in Site 1. An hGH mutant slightly enhanced in binding affinity for Site 1 and blocked in its ability to bind Site 2 was a better antagonist of the hGH receptor than the Site 2 mutant alone (Fuh et al., Science, 256:1677-1680 (1992).

Additional improvements in Site 1 affinity might be obtained by mutating more residues per library. However, it was not feasible to generate enough transformants to ensure that all possible residue combinations were represented when more than about five codons were randomized simultaneously (Lowman and Wells, Methods: Companion Methods Enzymol., 3:205-216 (1991)). Mutations at protein-protein interfaces usually exhibit additive effects upon binding (Wells, Biochemistry, 29:8509-8517 (1990)).

It has been disclosed that the lysine residues of hGH and bGH are involved in the interaction of hGH and bGH with somatotropic receptors, with the structure-function relationship particularly implicating the lysine or arginine residues at positions 41, 64, 70, and 115 (Martal et al., FEBS Lett., 180: 295-299 (1985)). Lysine residues were chemically modified by methylation, ethylation, guanidination, and acetimidination, resulting in reduced activity by radioreceptor assay.

Mutagenesis experiments on the binding surfaces between human growth hormone and its receptor have shown that a subset of contact side chains contribute a large fraction of the binding energy (Clackson and Wells, Science, 267:383-386 (1995)). In particular, Trp104 and Trp169 of the receptor each contribute more than 4.5 kcal mol⁻¹ in binding energy to the high-affinity (1:1) complex. This suggests that small-molecule mimics of the receptor surface, incorporating these energetically important contacts, might have significant affinity for hGH.

Acromegaly is a disease resulting from excess GH after puberty, when the long bones have fused characterized by bony overgrowth and soft tissue swelling as well as hypertrophy of internal organs, especially the heart. Acromegaly is typically caused by a pituitary tumor that secretes GH. The hallmarks of the disease are high levels of circulating GH and IGF-I.

Other growth hormone disorders characterized by elevated circulating levels of GH or of a mediator of GH action, include giantism, diabetes and its complications, such as, for instance, diabetic retinopathy and diabetic nephropathy, as well as vascular eye diseases that, like diabetic retinopathy, involve proliferative neovascularization. Examples of such eye diseases include, e.g. retinopathy of prematurity, retinopathy associated with sickle cell anemia, and age-related macular degeneration. Further disorders associated with GH are malignancies that grow in response to GH or a mediator of GH action (such as IGF-1) and malignancies that express GH receptors. Examples of such malignancies include Wilm's tumor, various sarcomas (e.g., osteogenic sarcoma), Burkitt's lymphoma, colorectal carcinoma, lung carcinoma, lymphoblastic leukemia, melanoma, and cancers of the breast, colon, prostate, thyroid, thymus, brain, salivary gland, bone, bone marrow and others.

Accordingly, it would be desirable to provide compounds which, upon administer to a patient, bind to and inhibit the activity of hGH.

### SUMMARY OF THE INVENTION

In one aspect of the invention, there is provided a method for inhibiting binding interaction between hGH or a mutant thereof and an hGH binding protein or receptor in a mammal comprising administering to said mammal an inhibiting amount of a compound of the general formula (I): 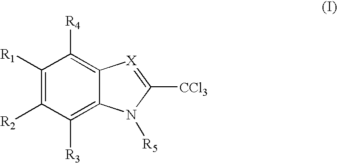
wherein
X is N or CH;
R₁ to R₄ are independently selected from the group consisting of H, halogen, hydroxyl, carboxyl, amino, nitro, alkyl, alkenyl, alkynyl, carbocycle, heterocycle; wherein said alkyl, alkenyl and alkynyl groups are optionally interrupted with N, O, S, SO, SO₂ or C(O) and optionally substituted with hydroxyl, halogen, carboxyl, amino, nitro, carbocycle or heterocycle; or R₁ and R₂ together form a five, six or seven member carbocycle or heterocycle optionally substituted with halogen, hydroxyl, carboxyl, amino or nitro; and
R₅ is selected from the group consisting of H, alkyl, alkenyl, alkynyl, carbocycle, heterocycle; wherein said alkyl, alkenyl and alkynyl groups are optionally interrupted with N, O, S, SO, SO₂ or C(O) and optionally substituted with a carbocycle or heterocycle.

### DETAILED DESCRIPTION OF THE INVENTION

The invention is directed to methods of inhibiting binding interaction between hGH or a mutant thereof and an hGH binding protein or receptor in a mammal comprising administering to said mammal an inhibiting amount of a compound of the general formula (I). Compounds of the invention are alternatively referred to herein as “inhibitors” or “antagonists”. The invention further includes treating diseases, conditions or disorders in which the inhibition of GH action provides therapeutic or prophylactic benefit. Such disorders include those in which a reduction of circulating levels of GH or of a mediator of GH action, such as IGF-I, is desirable, for example, disorders characterized by GH excess, such as giantism and acromegaly. Other examples include diabetes and its complications, such as, for instance, diabetic retinopathy and diabetic nephropathy, as well as vascular eye diseases that, like diabetic retinopathy, involve proliferative neovascularization. Examples of such eye diseases include, e.g., retinopathy of prematurity, retinopathy associated with sickle cell anemia, and age-related macular degeneration. Further disorders falling under the definition herein are malignancies that grow in response to GH or a mediator of GH action (such as IGF-1) and malignancies that express GH receptors. Examples of such malignancies include Wilm's tumor, various sarcomas (e.g., osteogenic sarcoma), Burkitt's lymphoma, colorectal carcinoma, lung carcinoma, lymphoblastic leukemia, melanoma, and cancers of the breast, colon, prostate, thyroid, thymus, brain, salivary gland, bone marrow, or bone. However, other cancers as defined below are also included herein. The preferred cancers for treatment herein are breast, prostate, colorectal, lung, and melanoma.

As used herein, “mammal” for purposes of treatment refers to any animal classified as a mammal, including humans, domestic, and farm animals, and zoo, sports, or pet animals, such as dogs, horses, cats, sheep, pigs, cows, etc. The preferred mammal herein is a human.

As used herein, the term “hyperglycemic disorders” refers to all forms of diabetes and disorders resulting from insulin resistance, such as Type I and Type II diabetes, as well as severe insulin resistance, hyperinsulinemia, and hyperlipidemia, e.g., obese subjects, and insulin-resistant diabetes, such as Mendenhall's Syndrome, Werner Syndrome, leprechaunism, lipoatrophic diabetes, and other lipoatrophies. The preferred hyperglycemic disorder is diabetes, especially Type 1 and Type II diabetes. “Diabetes” itself refers to a progressive disease of carbohydrate metabolism involving inadequate production or utilization of insulin and is characterized by hyperglycemia and glycosuria.

As used herein, the term “treating” refers to both therapeutic treatment and prophylactic or preventative measures. Those in need of treatment include those already with the disorder as well as those prone to having the disorder or diagnosed with the disorder or those in which the disorder is to be prevented. Consecutive treatment or administration refers to treatment on at least a daily basis without interruption in treatment by one or more days. Intermittent treatment or administration, or treatment or administration in an intermittent fashion, refers to treatment that is not consecutive, but rather cyclic in nature. The treatment regime herein can be either consecutive or intermittent.

The term “effective amount” refers to an amount of the inhibiting or antagonist compound required to reduce to treat the disorder or to reduce its symptoms in a mammal. In the case of cancer, the effective amount of the antagonist may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the antagonist may prevent growth and/or kill existing cancer cells, it may be cytostatic and/or cytotoxic. For cancer therapy, efficacy in vivo can, for example, be measured by assessing the time to disease progression (TTP) and/or determining the response rates (RR).

Compounds

Methods of the invention involve administration of compounds of formula (I) 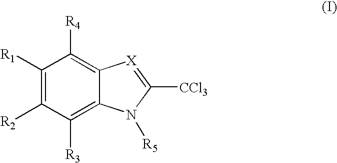
wherein X, R1, R2, R3, R4 and R5 are as described herein.

X is N or CH. In a preferred embodiment X is N.

R₁ and R₂ are independently selected from the group consisting of H, halogen, hydroxyl, carboxyl, amino (NH₂), nitro, SO₃, alkyl, alkenyl, alkynyl, carbocycle, heterocycle. The alkyl, alkenyl and alkynyl groups are linear or branched aliphatic chains up to 12 carbon atoms in length. In preferred embodiments the aliphatic chains are 1 to 8 carbon atoms in length and more preferable 1 to 4. Carbocycle groups are preferably from 3- to 7-membered which are saturated, unsaturated or partially unsaturated and are optionally substituted. Preferred carbocycles include cyclopropyl, cyclobutyl, cyclopentyl, cyclohexyl and phenyl. Heterocycles are preferably from 5- to 7-membered incorporating from 1 to 3 heteroatoms such as N, O and S and are saturated, unsaturated or partially unsaturated. Preferred heterocycles include pyridine, pyrazine, thiophene and triazine.

The aliphatic chains are optionally “interrupted” in that one or more carbon atoms in the chain are replaced with a heteroatom such as N (or NH), O, or S as well as SO, SO₂ or a carbonyl group i.e. C(O). Adjacent carbon atoms may be replaced to provide moieties such as amides i.e. —NH—C(O)— or —C(O)—NH—, sulfonamides i.e. —NH—SO₂— or —SO₂—NH—, esters i.e. —O—C(O)— or —C(O)—O—, thioesters i.e. —S—C(O)— or —C(O)—S—, ureas i.e. —NH—C(O)—NH—, amidines i.e. —NH—C(NH)— or —C(NH)—NH—, guanidines i.e. —NH—C(NH)—NH—, and others.

The aliphatic chains, carbocycles and heterocycles are optionally substituted with groups such as hydroxyl, halogen, carboxyl, amino, nitro, carbocycle or heterocycle. In a preferred embodiment R₁ and R₂ are independently selected from the group consisting of H, halo, nitro, carboxyl, alkyl, alkoxy and alkanoyl wherein said alkyl, alkoxy and alkanoyl are optionally substituted with halogen. In another preferred embodiment R₁ and R₂ are independently selected from the group consisting of H, F, Cl, Br, nitro, COOH, SO₃H, SO₂—Cl, SO₂—CF₃, SO₂—CHCl₂, Me, CF₃, OMe, O—CHF₂, O—CF₂—CHF₂, O—CH₂—CF₃, C(O)-nPr, C(O)NH₂, C(O)NH-Et-C(O)O-Me and Et-N(nPr)₂—In a particularly preferred embodiment R₁ and R₂ are independently H, Me or Cl. In other particularly preferred embodiments, while R₃ and R₄ are both H, R₁ and R₂ are both Cl, R₁ and R₂ are both Me, R₁ is Cl while R₂H, R₁ is Me while R₂ is H.

In an alternative embodiment R₁ and R₂ together with the carbon atoms from the benzene ring from which they depend, form a five, six or seven member carbocycle or heterocycle optionally substituted with halogen, hydroxyl, carboxyl, amino or nitro.

R₃ and R₄ are independently selected from the groups defined for R₁ and R₂. In preferred embodiments, R₃ and R₄ are independently H, halogen, alkyl, and nitro. In a particularly preferred embodiment R₃ are and R₄ are both H.

R₅ is selected from the group consisting of H, alkyl, alkenyl, alkynyl, carbocycle, heterocycle. The alkyl, alkenyl and alkynyl aliphatic chains are linear or branched and optionally interrupted and optionally substituted as described for R₁ and R₂. In a preferred embodiment R₅ is H, alkyl, a heterocycle or an alkyl substituted with a carbocycle or heterocycle. In particular embodiments, R₅ is H, Me, butyl, phenyl, benzyl, 4,6-dimethoxy-pyrimidin-2-yl, thiophenylmethyl, 4,6-dimethoxy-1,3,5-triazinyl, 4,6-diethoxy-1,3,5-triazinyl, p-hydroxyphenyl, p-chlorophenyl, and p-methylphenyl. In a particularly preferred embodiment R₅ is H. In another particularly preferred embodiment R₅ is Me. In another particularly preferred embodiment R₅ is Me while R₁ and R₂ are independently H, Me or Cl and R₃ and R₄ are both H.

In preferred embodiments, compounds employed in methods of the invention include: 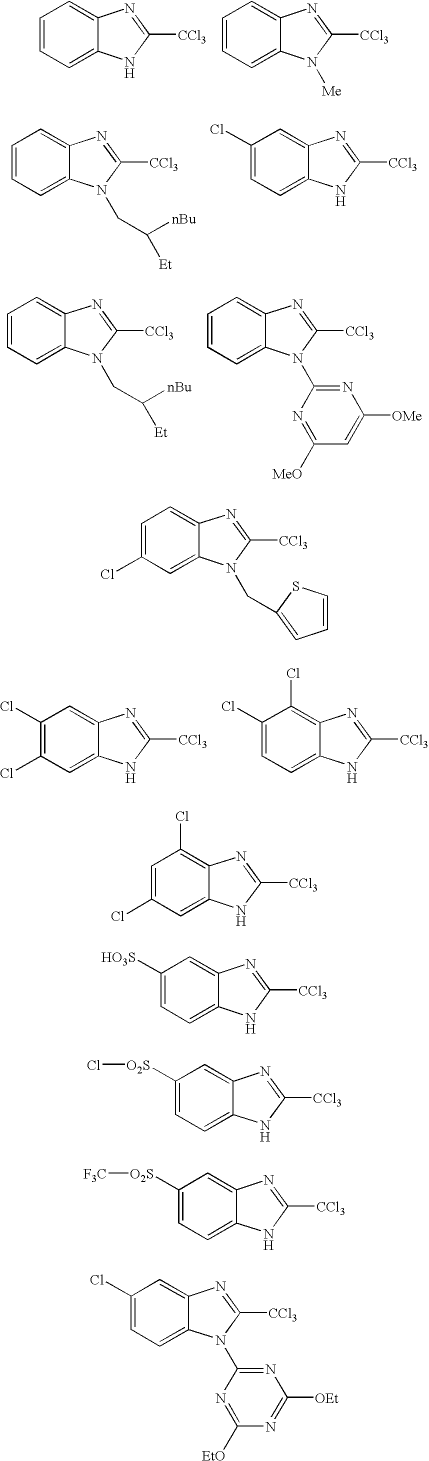 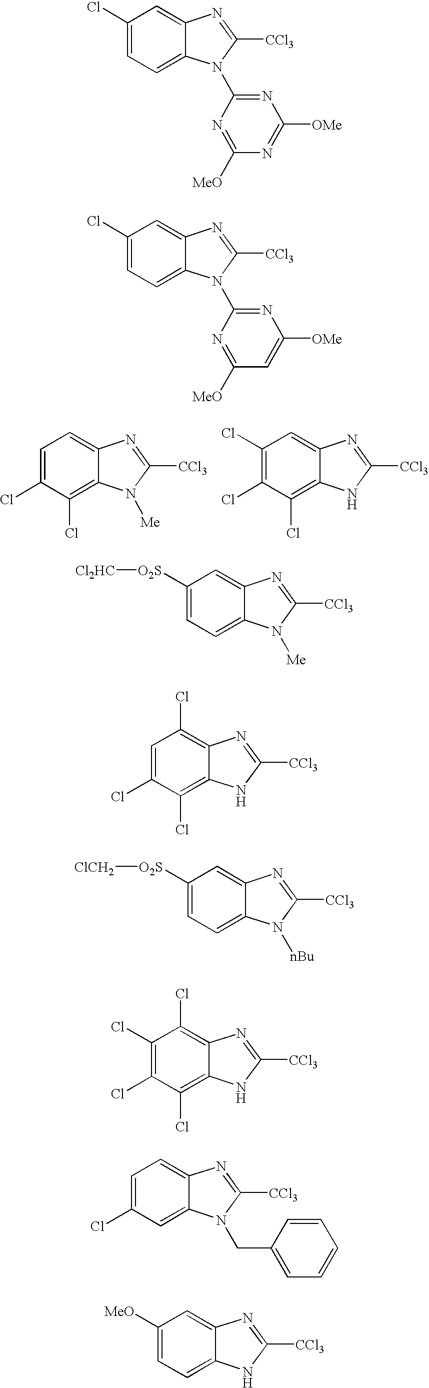 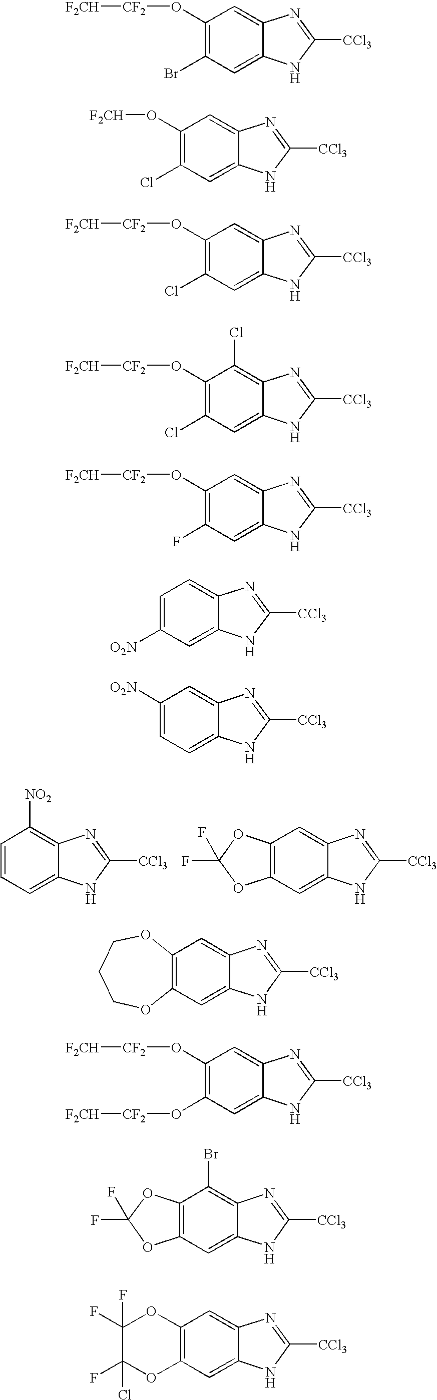 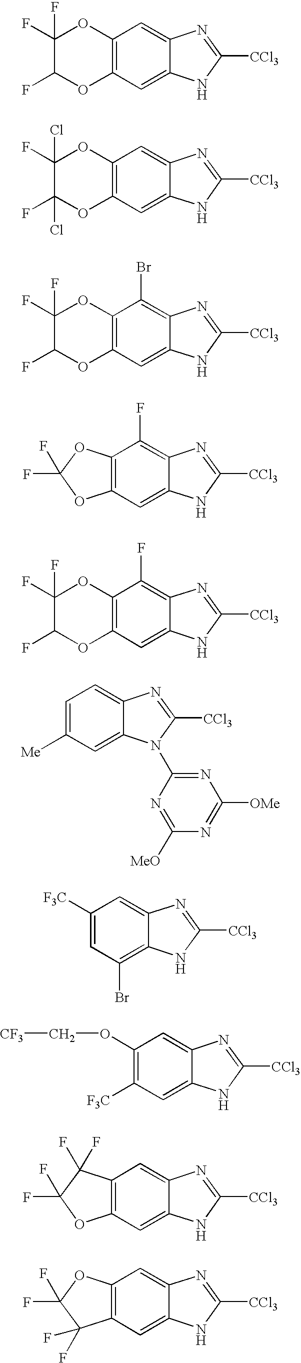 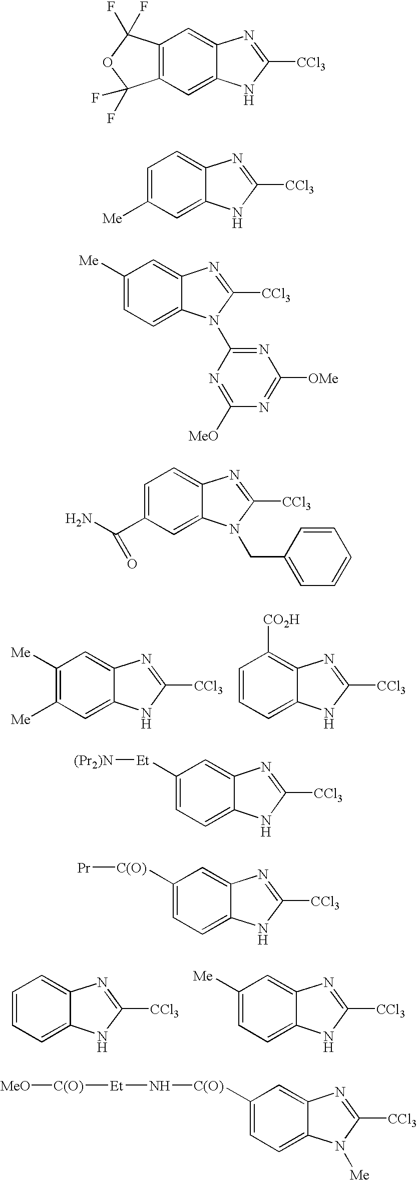
Synthesis of Compounds

Compounds employed in methods of the invention may be obtained commercially or prepared according to routine organic synthetic techniques from starting materials that are commercially available. Synthesis of a number of these compounds is described in Holan et al, J. Chem. Soc. C 1967, 1:20-5 as well as patent application publications GB29584, AU6886087 EP517476 and CA2115737. In general, compounds may be prepared according to the following general scheme:

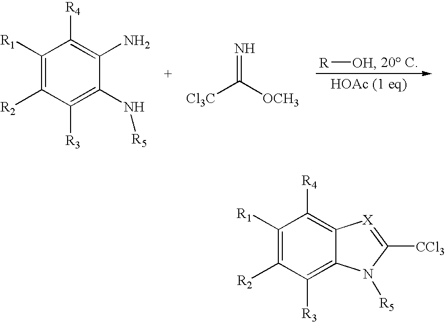

It will be appreciated that depending on the particular substituents present in the compound, suitable protection and deprotection procedures will be required as is standard in the art. Numerous protecting groups are described in Greene and Wuts, Protective Groups in Organic Chemistry, 2d edition, John Wiley and Sons, 1991, as well as detailed protection and deprotection procedures. For example, suitable amino protecting groups include t-butyloxycarbonyl (Boc), fluorenyl-methyloxycarbonyl (Fmoc), 2-trimethylsilyl-ethyoxycarbonyl (Teoc), 1-methyl-1-(4-biphenylyl)ethoxycarbonyl (Bpoc), allyloxycarbonyl (Alloc), and benzyloxycarbonyl (Cbz). Carboxyl groups can be protected as fluorenylmethyl groups and hydroxyl groups may be protected with trityl, monomethoxytrityl, dimethoxytrityl, and trimethoxytrityl groups.

It will be appreciated that compounds employed in methods of the invention may incorporate chiral centers and therefore exist as geometric and stereoisomers. All such isomers are contemplated and are within the scope of the invention whether in pure isomeric form or in mixtures of such isomers as well as racemates. Stereoisomeric compounds may be separated by established techniques in the art such as chromatography, i.e. chiral HPLC, or crystallization methods. Also tautomers of those compounds described herein are within the scope of the methods of the invention.

Salts

“Pharmaceutically acceptable” salts of compounds employed in methods of the invention include both acid and base addition salts. Pharmaceutically acceptable acid addition salt refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, salicyclic acid and the like.

Pharmaceutically acceptable base addition salts include those derived from inorganic bases such as sodium, potassium, lithium, ammonium, calcium, magnesium, iron, zinc, copper, manganese, aluminum salts and the like. Particularly preferred are the ammonium, potassium, sodium, calcium and magnesium salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

Additional Active Agents

Methods of the invention may further comprise administering additional active ingredients or agents such as a growth inhibitory agent, an angiostatic agent, or a cytotoxic agent. Preferably, the agent is a chemotherapeutic agent or antibody, preferably a growth-inhibitory antibody, an antibody that induces cell death, or an antibody that induces apoptosis.

Hence, the present application contemplates combining the antagonist with one or more other therapeutic agent(s), which depend on the particular indication being treated. The agent for example may be insulin if the indication is diabetes, or a cytotoxic agent for treating cancer.

If insulin is administered, it can be any formulation of insulin, but is preferably NPH insulin, and the dose of NPH insulin is from or about 5 to 50 units/injection (i.e., from or about 0.2 to 2 mg) twice a day subcutaneously. For a combination of insulin and the compound, the ratio of NPH insulin to compound in this formulation by weight is generally from or about 10:1 to 1:50, preferably from or about 1:1 to 1:20, more preferably from or about 1:1 to 1:10, still more preferably, from or about 1:1 to 1:5, and most preferably from or about 1:1 to 1:3.

Furthermore, the formulation is suitably administered along with an effective amount of a hypoglycemic agent such as a sulfonylurea. The hypoglycemic agent is administered to the mammal by any suitable technique including parenterally, intranasally, orally, or by any other effective route. Most preferably, the administration is by the oral route. For example, MICRONASE™ tablets (glyburide) marketed by Upjohn in 1.25, 2.5, and 5 mg tablet concentrations are suitable for oral administration. The usual maintenance dose for Type II diabetics, placed on this therapy, is generally in the range of from or about 1.25 to 20 mg per day, which may be given as a single dose or divided throughout the day as deemed appropriate. Physician's Desk Reference, 2563-2565 (1995). Other examples of glyburide-based tablets available for prescription include GLYNASE™ brand drug (Upjohn) and DIABETA™ brand drug (Hoechst-Roussel). GLUCOTROL™ (Pratt) is the trademark for a glipizide (1-cyclohexyl-3-(p-(2-(5-methylpyrazine carboxamide)ethyl)phenyl)sulfonyl)urea) tablet available in both 5- and 10-mg strengths and is also prescribed to Type II diabetics who require hypoglycemic therapy following dietary control or in patients who have ceased to respond to other sulfonylureas. Physician's Desk Reference, 1902-1903 (1995). Other hypoglycemic agents than sulfonylureas, such as the biguanides (e.g., metformin and phenformin) or thiazolidinediones (e.g., troglitozone), or other drugs affecting insulin action may also be employed. If a thiazolidinedione is employed with the compound, it is used at the same level as currently used or at somewhat lower levels, which can be adjusted for effects seen with the compound alone or together with the dione. The typical dose of troglitazone (REZULIN™) employed by itself is about 100-1000 mg per day, more preferably 200-800 mg/day, and this range is applicable herein. See, for example, Ghazzi et al., Diabetes, 46: 433-439 (1997). Other thiazolidinediones that are stronger insulin-sensitizing agents than troglitazone would be employed in lower doses.

The antagonist may be co-administered with a peptide (or multivalent antibodies), a monovalent or bivalent antibody (or antibodies), chemotherapeutic agent(s) (including cocktails of chemotherapeutic agents), other cytotoxic agent(s), anti-angiogenic agent(s), cytokines, and/or growth inhibitory agent(s). For instance, the antagonist may be combined with pro-apoptotic antibodies (e.g. bivalent or multivalent antibodies) directed against B-cell surface antigens (e.g. RITUXAN®, ZEVALIN® or BEXXAR® anti-CD20 antibodies) and/or with (1) pro-apoptotic antibodies (e.g. bivalent or multivalent antibodies directed against a receptor in the TNF receptor superfamily, such as anti-DR4 or anti-DR5 antibodies) or (2) cytokines in the TNF family of cytokines (e.g. Apo2L). Likewise, the antagonist may be administered along with anti-ErbB antibodies (e.g. HERCEPTIN® anti-HER2 antibody) alone or combined with (1) and/or (2). Alternatively, or additionally, the patient may receive combined radiation therapy (e.g. external beam irradiation or therapy with a radioactive labeled agent, such as an antibody), ovarian ablation, chemical or surgical, or high-dose chemotherapy along with bone marrow transplantation or peripheral-blood stem-cell rescue or transplantation. Such combined therapies noted above include combined administration (where the two or more agents are included in the same or separate formulations) and separate administration, in which case, administration of the antagonist can occur prior to, and/or following, administration of the adjunct therapy or therapies. The effective amount of such other agents depends on the amount of antagonist present in the formulation, the type of disorder or treatment, and other factors discussed above. These are generally used in the same dosages and with administration routes as used hereinbefore or about from 1 to 99% of the heretofore employed dosages.

The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu) chemotherapeutic agents, and toxins such as small molecule toxins or enzymatically active toxins of bacterial, fungal, plant or animal origin, including fragments and/or variants thereof.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylene-thiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin γ₁^{I} and calicheamicin θ^{I}₁, see, e.g., Agnew, Chem Intl. Ed. Engl., 33:183-186 (1994); dynemicin, including dynemicin A; bisphosphonates, such as clodronate; an esperamicin; as well as neocarzinostatin chromophore and related chromoprotein enediyne antiobiotic chromomophores), aclacinomysins, actinomycin, authramycin, azaserine, bleomycins, cactinomycin, carabicin, carminomycin, carzinophilin, chromomycins, dactinomycin, daunorubicin, detorubicin, 6-diazo-5-oxo-L-norleucine, doxorubicin (Adriamycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK®; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitoxantrone; vincristine; vinorelbine (Navelbine™); novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including Nolvadex™), raloxifene, droloxifene, 4-hydroxytamoxifen, trioxifene, keoxifene, LY117018, onapristone, and toremifene (Fareston™); aromatase inhibitors that inhibit the enzyme aromatase, which regulates estrogen production in the adrenal glands, such as, for example, 4(5)-imidazoles, aminoglutethimide, megestrol acetate (Megace™), exemestane (Aromasin™), formestane, fadrozole, vorozole (Rivisor™), letrozole (Femara™), and anastrozole (Arimidex™); and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signalling pathways implicated in abherant cell proliferation, such as, for example, PKC-alpha, Raf, and H-Ras; ribozymes such as a VEGF expression inhibitor (e.g. Angiozyme™) and a HER2 expression inhibitor; vaccines such as gene therapy vaccines, for example, Allovectin™, Leuvectin™, and Vaxid™; Proleukin™ (rIL-2); Lurtotecan (a topoisomerase I inhibitor); Abarelix™ (rGnRH); and pharmaceutically acceptable salts, acids or derivatives of any of the above. A “growth inhibitory agent” when used herein refers to a compound or composition which inhibits growth of a cell in vitro and/or in vivo. Thus, the growth inhibitory agent may be one that significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (W B Saunders: Philadelphia, 1995), especially p. 13.

Examples of “growth inhibitory” anti-HER2 antibodies are those which bind to HER2 and inhibit the growth of cancer cells overexpressing HER2. Preferred growth inhibitory anti-HER2 antibodies inhibit growth of SKBR3 breast tumor cells in cell culture by greater than 20%, and preferably greater than 50% (e.g. from about 50% to about 100%) at an antibody concentration of about 0.5 to 30 μg/ml, where the growth inhibition is determined six days after exposure of the SKBR3 cells to the antibody (see U.S. Pat. No. 5,677,171 issued Oct. 14, 1997).

An antibody which “induces cell death” is one which causes a viable cell to become nonviable. The cell is generally one which expresses the antigen to which the antibody binds, especially where the cell overexpresses the antigen. Preferably, the cell is a cancer cell, e.g. a breast, ovarian, stomach, endometrial, salivary gland, lung, kidney, colon, thyroid, pancreatic or bladder cell. In vitro, the cell may be a SKBR3, BT474, Calu 3, MDA-MB-453, MDA-MB-361 or SKOV3 cell. Cell death in vitro may be determined in the absence of complement and immune effector cells to distinguish cell death induced by antibody dependent cell-mediated cytotoxicity (ADCC) or complement dependent cytotoxicity (CDC). Thus, the assay for cell death may be performed using heat inactivated serum (i.e. in the absence of complement) and in the absence of immune effector cells. To determine whether the antibody is able to induce cell death, loss of membrane integrity as evaluated by uptake of propidium iodide (PI), trypan blue (see Moore et al. Cytotechnology, 17:1-11 (1995)) or 7AAD can be assessed relative to untreated cells.

An antibody that “induces apoptosis” is one which induces programmed cell death as determined by binding of annexin V, fragmentation of DNA, cell shrinkage, dilation of endoplasmic reticulum, cell fragmentation, and/or formation of membrane vesicles (called apoptotic bodies) The cell is one which expresses the antigen to which the antibody binds and may be one which overexpresses the antigen. The cell may be a tumor cell, e.g. a breast, ovarian, stomach, endometrial, salivary gland, lung, kidney, colon, thyroid, pancreatic or bladder cell. In vitro, the cell may be a SKBR3, BT474, Calu 3 cell, MDA-MB-453, MDA-MB-361 or SKOV3 cell. Various methods are available for evaluating the cellular events associated with apoptosis. For example, phosphatidyl serine (PS) translocation can be measured by annexin binding; DNA fragmentation can be evaluated through DNA laddering as disclosed in the example herein; and nuclear/chromatin condensation along with DNA fragmentation can be evaluated by any increase in hypodiploid cells. Preferably, the antibody which induces apoptosis is one which results in about 2 to 50 fold, preferably about 5 to 50 fold, and most preferably about 10 to 50 fold, induction of annexin binding relative to untreated cell in an annexin binding assay using cells expressing the antigen to which the antibody binds.

Examples of antibodies that induce apoptosis include the anti-HER2 monoclonal antibodies 7F3 (ATCC HB-12216), and 7C2 (ATCC HB 12215), including humanized and/or affinity matured variants thereof; the anti-DR5 antibodies 3F11.39.7 (ATCC HB-12456); 3H3.14.5 (ATCC HB-12534); 3D5.1.10 (ATCC HB-12536); and 3H3.14.5 (ATCC HB-12534), including humanized and/or affinity matured variants thereof; the human anti-DR5 receptor antibodies 16E2 and 20E6, including affinity matured variants thereof (WO98/51793, expressly incorporated herein by reference); the anti-DR4 antibodies 4E7.24.3 (ATCC HB-12454); 4H6.17.8 (ATCC HB-12455); 1H5.25.9 (ATCC HB-12695); 4G7.18.8 (ATCC PTA-99); and 5G11.17.1 (ATCC HB-12694), including humanized and/or affinity matured variants thereof.

Administration Routes

The actual amount of compound administered and the route of administration will depend upon the particular disease or condition as well as other factors such as the size, age, sex and ethnic origin of the individual being treated and is determined by routine analysis. In methods of the invention, the compound may be administered orally (including buccal, sublingual, inhalation), nasally, rectally, vaginally, intravenously, intradermally, subcutaneously and topically.

Formulations

Compounds will be formulated into compositions suitable for administration for example with suitable carriers, diluents, thickeners, adjuvants etc. as are routine in the formulation art. Accordingly, another aspect of the invention provides pharmaceutical compositions comprising a compound of formula (I) and a pharmaceutically acceptable carrier, excipient or adjuvant.

Compositions of the invention may also include additional active ingredients. Dosage forms include solutions, powders, tablets, capsules, gel capsules, suppositories, topical ointments and creams and aerosols for inhalation. Formulations for non-parenteral administration may include sterile aqueous solutions which may also contain buffers, diluents and other suitable additives. Pharmaceutically acceptable organic or inorganic carrier substances suitable for non-parenteral administration which do not deleteriously react with compounds of the invention can be used. Suitable pharmaceutically acceptable carriers include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like. The formulations can be sterilized and, if desired, mixed with auxiliary agents, e.g., lubricants, preservatives, stabilizers, wetting agents, emulsifiers, salts for influencing osmotic pressure, buffers, colorings flavorings and/or aromatic substances and the like which do not deleteriously react with compounds of the invention. Aqueous suspensions may contain substances which increase the viscosity of the suspension including, for example, sodium carboxymethylcellulose, sorbitol and/or dextran. Optionally, the suspension may also contain stabilizers.

In a preferred embodiment, compounds of the invention are administered via oral delivery. Compositions for oral administration include powders or granules, suspensions or solutions in water or non-aqueous media, capsules, sachets, troches, tablets or SECs (soft elastic capsules or caplets). Thickeners, flavoring agents, diluents, emulsifiers, dispersing aids, carrier substances or binders may be desirably added to such formulations. Such formulations may be used to effect delivering the compounds to the alimentary canal for exposure to the mucosa thereof. Accordingly, the formulation can consist of material effective in protecting the compound from pH extremes of the stomach, or in releasing the compound over time, to optimize the delivery thereof to a particular mucosal site. Enteric coatings for acid-resistant tablets, capsules and caplets are known in the art and typically include acetate phthalate, propylene glycol and sorbitan monoleate.

Various methods for producing formulations for alimentary delivery are well known in the art. See, generally Remington's Pharmaceutical Sciences, 18th Ed., Gennaro, ed., Mack Publishing Co., Easton, Pa., 1990. The formulations of the invention can be converted in a known manner into the customary formulations, such as tablets, coated tablets, pills, granules, aerosols, syrups, emulsions, suspensions and solutions, using inert, non-toxic, pharmaceutically suitable excipients or solvents. The therapeutically active compound should in each case be present in a concentration of about 0.5% to about 99% by weight of the total mixture, that is to say in amounts which are sufficient to achieve the desired dosage range. The formulations are prepared, for example, by extending the active compounds with solvents and/or excipients, if appropriate using emulsifying agents and/or dispersing agents, and, for example, in the case where water is used as the diluent, organic solvents can be used as auxiliary solvents if appropriate.

Compositions may also be formulated with binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrates (e.g., starch or sodium starch glycolate); or wetting agents (e.g., sodium lauryl sulfate). Tablets may be coated by methods well known in the art. The preparations may also contain flavoring, coloring and/or sweetening agents as appropriate.

Formulations of the present invention suitable for oral administration may be presented as discrete units such as capsules, cachets or tablets each containing predetermined amounts of the active ingredients; as powders or granules; as solutions or suspensions in an aqueous liquid or a non-aqueous liquid; or as oil-in-water emulsions or water-in-oil liquid emulsions. A tablet may be made by compression or molding, optionally with one or more accessory ingredients. Compressed tablets may be prepared by compressing in a suitable machine, the active ingredients in a free-flowing form such as a powder or granules, optionally mixed with a binder, lubricant, inert diluent, preservative, surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredients therein.

Kits

In another embodiment of the invention, an article of manufacture or kit containing materials useful for the treatment of the disorders described above is provided. The article of manufacture comprises a container and instructions, such as a label or package or product insert on or associated with the container. Suitable containers include, for example, bottles, vials, syringes, etc., preferably a vial. The containers may be formed from a variety of materials such as glass or plastic. The container holds a composition with at least the antagonist herein and may have a sterile access port (for example the container may be an intravenous solution bag or a vial having a stopper pierceable by a hypodermic injection needle). The instructions direct the user how to utilize the composition for treating the condition of choice, such as cancer. The kit may optionally include a second container with a composition comprising a further active agent as set forth above, such as a cytotoxic agent. Alternatively, or additionally, the article of manufacture may further comprise a second (or third) container comprising a pharmaceutically acceptable buffer, such as bacteriostatic water for injection (BWFI), phosphate-buffered saline, Ringer's solution, and dextrose solution. It may further include other materials desirable from a commercial and user standpoint, including other buffers, diluents, filters, needles, and syringes.

### EXAMPLE 1

The following is presented by way of example and is not to be construed as a limitation to the scope of the invention. All citations used herein are expressly incorporated herein by reference.

A collection of heterocyclic aromatic compounds (384 total; 1 mmol each) was obtained from Aldrich Chemical Company or prepared as described in Holan et al., J. Chem Soc. C, 20-25 (1967)) incorporated herein by reference. Each compound was dissolved in dimethyl sulfoxide (DMSO) to yield a 100-mM solution and arrayed in 96-well polypropylene deep-well plates (Beckman). Dilutions (10-fold in DMSO) were prepared from the parent plates; these 10-mM stocks were then diluted 50-fold in the screening assay as described below (200 μM final concentration; 2% DMSO).

Human growth hormone (100 μM) in phosphate-buffered saline (PBS) was treated with 2 molar equivalents of EZ-Link Sulfo-NHS-LC-biotin™ (Pierce 21335™) according to the manufacturer's instructions. Binding of the biotinylated human growth hormone (b-hGH) to the extracellular domain of its receptor was assayed in an ELISA format. Briefly, Nunc Maxi-Sorb™ 96-well plates were treated with a solution of the receptor (2 μg/mL) in phosphate-buffered saline (PBS) overnight at 4° C. Plates were then blocked with a 0.2% solution of bovine serum albumin (BSA; Sigma A-7638) in PBS for 2 hours at room temperature. An appropriate dilution of b-hGH (generally about 1.5 nM final concentration in PBS containing 0.05% Tween-20 T detergent) was added to polypropylene plates containing aliquots of the screening collection (147 μL b-hGH to 3 μL compound in DMSO) or DMSO alone, and the mixtures were allowed to equilibrate at room temperature for approximately 45 min. These mixtures were then transferred to the receptor-coated plates and allowed to stand for 15 min. Plates were washed (10 times) with PBS/Tween-20™. Streptavidin-HRP conjugate (Zymed Laboratories 43-4323™) followed by TMB peroxidase reagent (Kirkegaard & Perry Laboratories 50-76-03™) was used to detect bound b-hGH. Several potential inhibitor compounds were re-tested at multiple concentrations. Table 1 below provides IC50 of the compounds tested.

Inhibition curves for several of compounds were determined. The 5-chlorobenzimidazoles lacking the 2-trichloromethyl group did not inhibit the binding of b-hGH to receptor, whereas all compounds tested with the trichloromethyl group did inhibit. The benzimidazole core structure was also important for inhibition: neither 2-trichloromethyl-substituted benzoxazole or benzothiazole was able to block binding at the concentrations tested (table 1).

TABLE 1

| compd no. | structure | IC₅₀ (μM) |
|---|---|---|
| (i) | 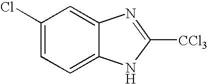 | 77 |
| (ii) | 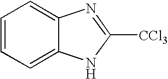 | 170 |
| (iii) | 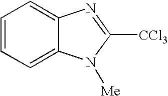 | 150 |
| (iv) | 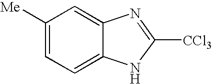 | 98 |
| (v) | 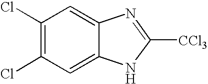 | 51 |
| (vi) | 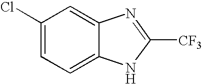 | >1000 |
| (vii) | 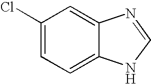 | >1000 |
| (viii) | 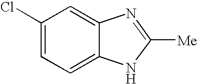 | >1000 |
| (ix) | 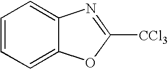 | >1000 |
| (x) | 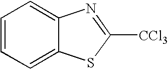 | >1000 |
| (xi) | 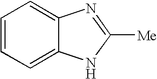 | >1000 |
| (xii) | 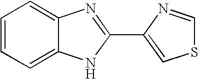 | >1000 |
| (xiii) | 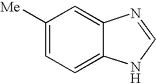 | >1000 |

What is claimed is:

1. A method for inhibiting binding interaction between hGH or a mutant thereof and an hGH binding protein or receptor in a mammal comprising administering to said mammal an inhibiting amount of a compound of the general formula (I): 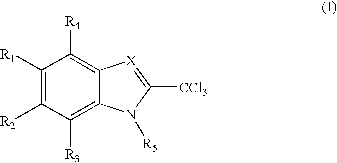
wherein (I)
X is N or CH;
R₁ to R₄ are independently selected from the group consisting of H, halogen, hydroxyl, carboxyl, amino, nitro, SO₃, alkyl, alkenyl, alkynyl, carbocycle, heterocycle; wherein said alkyl, alkenyl and alkynyl groups are optionally interrupted with N, O, S, SO, SO₂ or C(O) and optionally substituted with hydroxyl, halogen, carboxyl, amino, nitro, carbocycle or heterocycle; or
R₁ and R₂ together form a five, six or seven member carbocycle or heterocycle optionally substituted with halogen, hydroxyl, carboxyl, amino or nitro; and
R₅ is selected from the group consisting of H, alkyl, alkenyl, alkynyl, carbocycle, heterocycle; wherein said alkyl, alkenyl and alkynyl groups are optionally interrupted with N, O, S, SO, SO₂ or C(O) and optionally substituted with a carbocycle or heterocycle.

2. The method according to claim 1, wherein X is N.

3. The method according to claim 1, wherein R₁, R₂, R₃ and R₄ are independently H, halo, nitro, carboxyl, alkyl, alkoxy and alkanoyl wherein said alkyl, alkoxy and alkanoyl are optionally substituted with halogen.

4. The method according to claim 1, wherein R₁, R₂, R₃ and R₄ are independently selected from the group consisting of H, F, Cl, Br, nitro, COOH, SO₃H, SO₂—Cl, SO₂—CF₃, SO₂—CHCl₂, Me, CF₃, OMe, O—CHF₂, b-CF₂—CHF₂, O—CH₂—CF₃, C(O)-nPr, C(O)NH₂, C(O)NH-Et-C(O)O—Me and Et-N(nPr)₂.

5. The method according to claim 1, wherein R₁ and R₂ are independently H, Me or Cl while R₃ and R₄ are both H.

6. The method according to claim 1, wherein R₅ is H, alkyl, aryl or aralkyl.

7. The method according to claim 1, wherein R₅H.

8. The method according to claim 1, wherein R₅ is Me.

9. The method according to claim 1, wherein said compound of formula (I) is selected from the group consisting of:

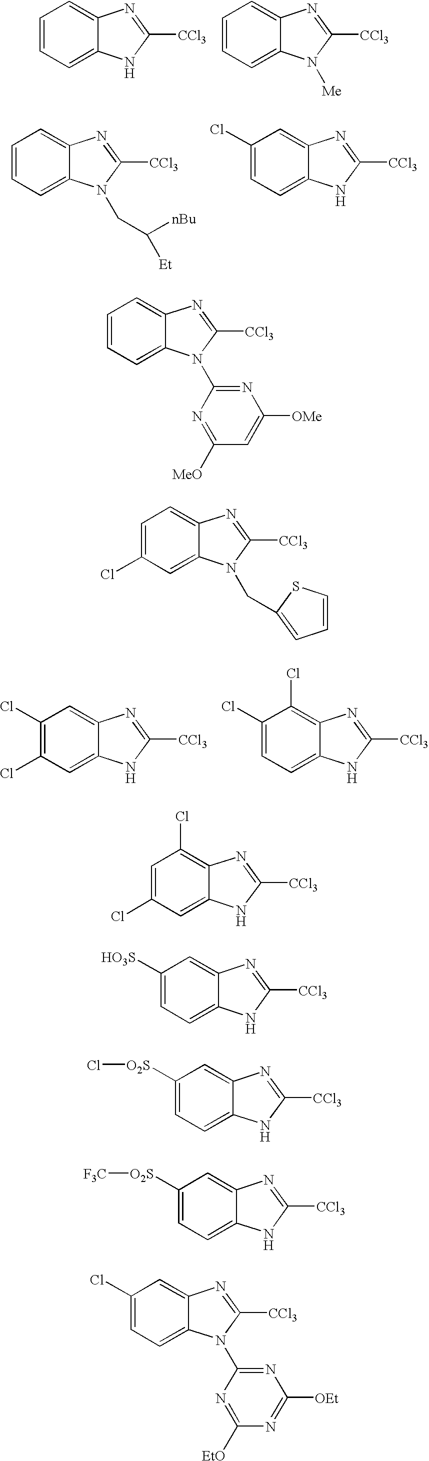

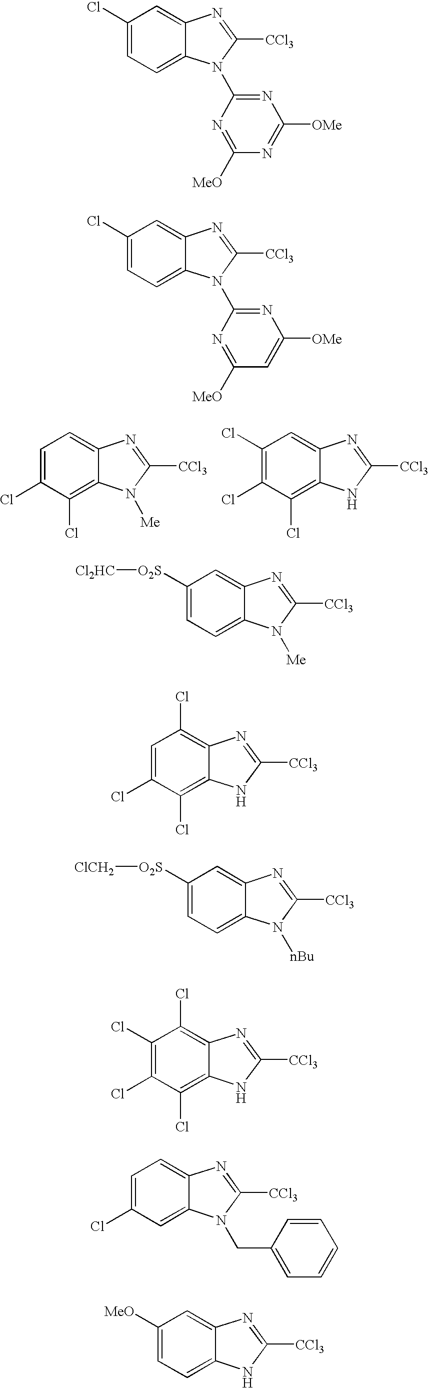

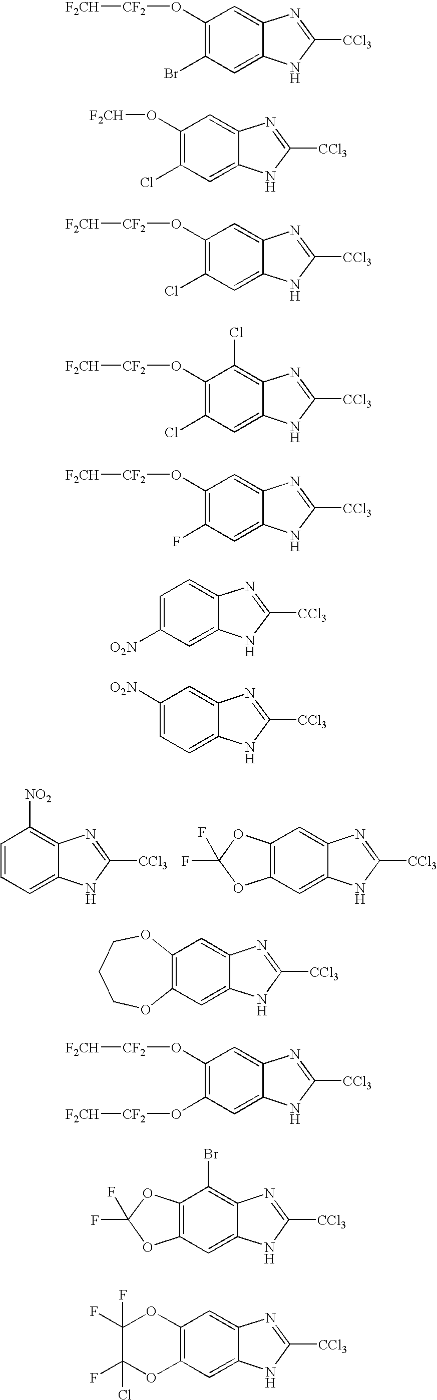

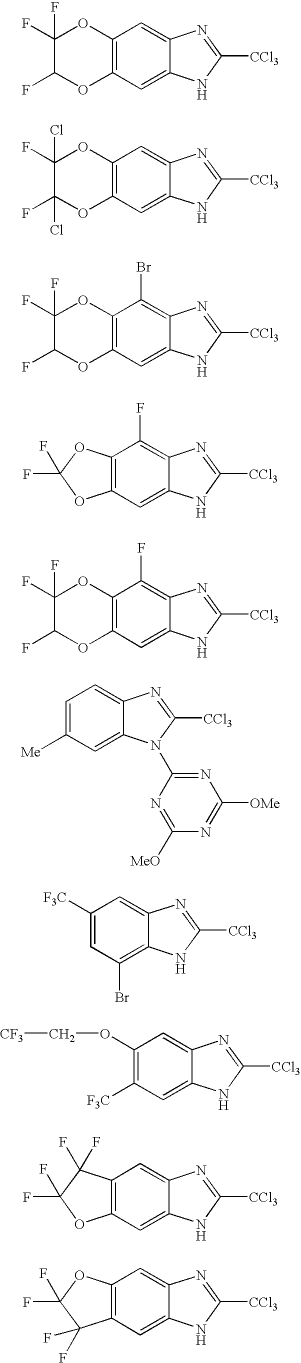

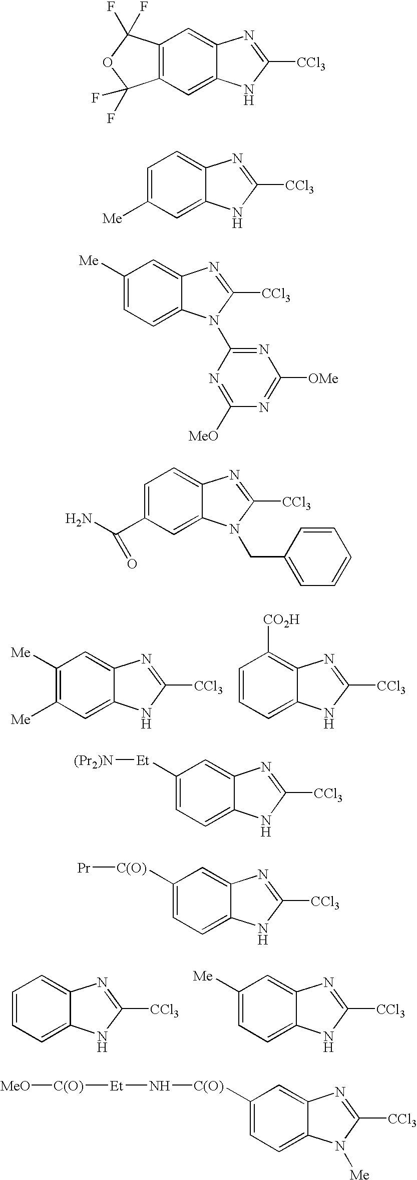

10. A method of treating a disease or condition in a mammal associated with an excess of hGH or hGH receptor comprising administering to said mammal an effective amount of a compound of formula (I) 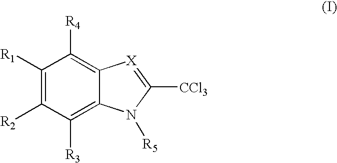
wherein (I)
X is N or CH;
R₁ to R₄ are independently selected from the group consisting of H, halogen, hydroxyl, carboxyl, amino, nitro, alkyl, alkenyl, alkynyl, carbocycle, heterocycle; wherein said alkyl, alkenyl and alkynyl groups are optionally interrupted with N, O, S, SO, SO₂ or C(O) and optionally substituted with hydroxyl, halogen, carboxyl, amino, nitro, carbocycle or heterocycle; or
R₁ and R₂ together form a five, six or seven member carbocycle or heterocycle optionally substituted with halogen, hydroxyl, carboxyl, amino or nitro; and
R₅ is selected from the group consisting of H, alkyl, alkenyl, alkynyl, carbocycle, heterocycle; wherein said alkyl, alkenyl and alkynyl groups are optionally interrupted with N, O, S, SO, SO₂ or C(O) and optionally substituted with a carbocycle or heterocycle.

11. The method according to claim 10, wherein said disease or condition is selected from the group consisting of cancer, a hypoglycemic disorder, diabetes, giantism, acromegaly, age-related macular degeneration, diabetic neuropathy, or diabetic retinopathy.

12. The method according to claim 11, wherein said cancer is breast cancer, prostate cancer, colorectal cancer, lung cancer, or melanoma.

13. The method according to claim 10, wherein said mammal is a human.

14. The method according to claim 10, wherein said compound is selected from the group consisting of:

15. The method according to claim 10, further comprising administering to said mammal a second agent effective in treating said disease or condition.

16. The method according to claim 15, wherein said second agent is insulin, a hypoglycemic agent, a growth inhibitory agent, an angiostatic agent, or a cytotoxic agent.

17. The method according to claim 15, wherein said second agent is a chemotherapeutic agent.
